(19) United States
(12) Patent Application Publication
(10) Pub. No.: US 2024/0079137 A1
(43) Pub. Date: Mar. 7, 2024

(54) SYSTEM AND METHOD FOR STRESS PROFILING AND PERSONALIZED STRESS INTERVENTION RECOMMENDATION

(71) Applicant: Samsung Electronics Co., Ltd., Suwon-si (KR)

(72) Inventors: Viswam Nathan, Clovis, CA (US); Md Mahbubur Rahman, San Jose, CA (US); Jilong Kuang, San Jose, CA (US); Jun Gao, Menlo Park, CA (US)

(21) Appl. No.: 17/930,017

(22) Filed: Sep. 6, 2022

(51) Int. Cl. G16H 50/20; G16H 40/67

(52) U.S. Cl. CPC G16H 50/20; G16H 40/67

(57) ABSTRACT

A method includes receiving stress-related measurements collected by one or more stress sensors, where the stress-related measurements represent one or more physiological responses of a user to a stressor. The method also includes receiving context data collected by one or more context sensors, where the context data represents a context associated with the user. The method further includes determining stress profile features associated with the user based on the stress-related measurements. The method also includes providing the stress profile features to a trained stress profile identification machine learning model to select a stress profile from among multiple candidate stress profiles for association with the user. The method further includes providing the selected stress profile and the context data to a trained stress intervention recommendation machine learning model to select a stress intervention activity for the user. In addition, the method includes recommending the selected stress intervention activity to the user.

### TECHNICAL FIELD

This disclosure relates generally to health and wellness systems. More specifically, this disclosure relates to a system and method for stress profiling and personalized stress intervention recommendation.

### BACKGROUND

Stress is a leading cause of physical and psychological conditions in modern life. In the United States, 77% of people report regularly experiencing physical symptoms caused by stress, and 73% of people regularly experience psychological symptoms caused by stress. Among other medical issues, unchecked stress is associated with brain function complications, compromised immune system functions, and cardiovascular and gastrointestinal complications. The annual cost of stress-related healthcare and lost productivity in the United States is currently estimated to be $300 billion. Stressors can come from a variety of different sources (such as social stress, deadline stress, traffic stress, and the like), and a given individual can have a unique set of responses to these stressors. Many individuals may not be fully aware of the context and extent of the stresses they undergo.

### SUMMARY

This disclosure provides a system and method for stress profiling and personalized stress intervention recommendation.

In a first embodiment, a method includes receiving stress-related measurements collected by one or more stress sensors, where the stress-related measurements represent one or more physiological responses of a user to a stressor. The method also includes receiving context data collected by one or more context sensors, where the context data represents a context associated with the user. The method further includes determining stress profile features associated with the user based on the stress-related measurements. The method also includes providing the stress profile features to a trained stress profile identification machine learning model to select a stress profile from among multiple candidate stress profiles for association with the user. The method further includes providing the selected stress profile and the context data to a trained stress intervention recommendation machine learning model to select a stress intervention activity for the user. In addition, the method includes recommending the selected stress intervention activity to the user.

In a second embodiment, an electronic device includes at least one memory configured to store instructions. The electronic device also includes at least one processing device configured when executing the instructions to receive stress-related measurements collected by one or more stress sensors, where the stress-related measurements represent one or more physiological responses of a user to a stressor. The at least one processing device is also configured when executing the instructions to receive context data collected by one or more context sensors, where the context data represents a context associated with the user. The at least one processing device is further configured when executing the instructions to determine stress profile features associated with the user based on the stress-related measurements. The at least one processing device is also configured when executing the instructions to provide the stress profile features to a trained stress profile identification machine learning model to select a stress profile from among multiple candidate stress profiles for association with the user. The at least one processing device is further configured when executing the instructions to provide the selected stress profile and the context data to a trained stress intervention recommendation machine learning model to select a stress intervention activity for the user. In addition, the at least one processing device is configured when executing the instructions to recommend the selected stress intervention activity to the user.

In a third embodiment, a non-transitory machine-readable medium contains instructions that when executed cause at least one processor of an electronic device to receive stress-related measurements collected by one or more stress sensors, where the stress-related measurements represent one or more physiological responses of a user to a stressor. The medium also contains instructions that when executed cause the at least one processor to receive context data collected by one or more context sensors, where the context data represents a context associated with the user. The medium further contains instructions that when executed cause the at least one processor to determine stress profile features associated with the user based on the stress-related measurements. The medium also contains instructions that when executed cause the at least one processor to provide the stress profile features to a trained stress profile identification machine learning model to select a stress profile from among multiple candidate stress profiles for association with the user. The medium further contains instructions that when executed cause the at least one processor to provide the selected stress profile and the context data to a trained stress intervention recommendation machine learning model to select a stress intervention activity for the user. In addition, the medium contains instructions that when executed cause the at least one processor to recommend the selected stress intervention activity to the user.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

As used here, terms and phrases such as “have,” “may have,” “include,” or “may include” a feature (like a number, function, operation, or component such as a part) indicate the existence of the feature and do not exclude the existence of other features. Also, as used here, the phrases “A or B,” “at least one of A and/or B,” or “one or more of A and/or B” may include all possible combinations of A and B. For example, “A or B,” “at least one of A and B,” and “at least one of A or B” may indicate all of (1) including at least one A, (2) including at least one B, or (3) including at least one A and at least one B. Further, as used here, the terms “first” and “second” may modify various components regardless of importance and do not limit the components. These terms are only used to distinguish one component from another. For example, a first user device and a second user device may indicate different user devices from each other, regardless of the order or importance of the devices. A first component may be denoted a second component and vice versa without departing from the scope of this disclosure.

It will be understood that, when an element (such as a first element) is referred to as being (operatively or communicatively) “coupled with/to” or “connected with/to” another element (such as a second element), it can be coupled or connected with/to the other element directly or via a third element. In contrast, it will be understood that, when an element (such as a first element) is referred to as being “directly coupled with/to” or “directly connected with/to” another element (such as a second element), no other element (such as a third element) intervenes between the element and the other element.

As used here, the phrase “configured (or set) to” may be interchangeably used with the phrases “suitable for,” “having the capacity to,” “designed to,” “adapted to,” “made to,” or “capable of” depending on the circumstances. The phrase “configured (or set) to” does not essentially mean “specifically designed in hardware to.” Rather, the phrase “configured to” may mean that a device can perform an operation together with another device or parts. For example, the phrase “processor configured (or set) to perform A, B, and C” may mean a generic-purpose processor (such as a CPU or application processor) that may perform the operations by executing one or more software programs stored in a memory device or a dedicated processor (such as an embedded processor) for performing the operations.

The terms and phrases as used here are provided merely to describe some embodiments of this disclosure but not to limit the scope of other embodiments of this disclosure. It is to be understood that the singular forms “a,” “an,” and “the” include plural references unless the context clearly dictates otherwise. All terms and phrases, including technical and scientific terms and phrases, used here have the same meanings as commonly understood by one of ordinary skill in the art to which the embodiments of this disclosure belong. It will be further understood that terms and phrases, such as those defined in commonly-used dictionaries, should be interpreted as having a meaning that is consistent with their meaning in the context of the relevant art and will not be interpreted in an idealized or overly formal sense unless expressly so defined here. In some cases, the terms and phrases defined here may be interpreted to exclude embodiments of this disclosure.

Examples of an “electronic device” according to embodiments of this disclosure may include at least one of a smartphone, a tablet personal computer (PC), a mobile phone, a video phone, an e-book reader, a desktop PC, a laptop computer, a netbook computer, a workstation, a personal digital assistant (PDA), a portable multimedia player (PMP), an MP3 player, a mobile medical device, a camera, or a wearable device (such as smart glasses, a head-mounted device (HMD), electronic clothes, an electronic bracelet, an electronic necklace, an electronic accessory, an electronic tattoo, a smart mirror, or a smart watch). Other examples of an electronic device include a smart home appliance. Examples of the smart home appliance may include at least one of a television, a digital video disc (DVD) player, an audio player, a refrigerator, an air conditioner, a cleaner, an oven, a microwave oven, a washer, a drier, an air cleaner, a set-top box, a home automation control panel, a security control panel, a TV box (such as SAMSUNG HOMESYNC, APPLETV, or GOOGLE TV), a smart speaker or speaker with an integrated digital assistant (such as SAMSUNG GALAXY HOME, APPLE HOMEPOD, or AMAZON ECHO), a gaming console (such as an XBOX, PLAYSTATION, or NINTENDO), an electronic dictionary, an electronic key, a camcorder, or an electronic picture frame. Still other examples of an electronic device include at least one of various medical devices (such as diverse portable medical measuring devices (like a blood sugar measuring device, a heartbeat measuring device, or a body temperature measuring device), a magnetic resource angiography (MRA) device, a magnetic resource imaging (MRI) device, a computed tomography (CT) device, an imaging device, or an ultrasonic device), a navigation device, a global positioning system (GPS) receiver, an event data recorder (EDR), a flight data recorder (FDR), an automotive infotainment device, a sailing electronic device (such as a sailing navigation device or a gyro compass), avionics, security devices, vehicular head units, industrial or home robots, automatic teller machines (ATMs), point of sales (POS) devices, or Internet of Things (IoT) devices (such as a bulb, various sensors, electric or gas meter, sprinkler, fire alarm, thermostat, street light, toaster, fitness equipment, hot water tank, heater, or boiler). Other examples of an electronic device include at least one part of a piece of furniture or building/structure, an electronic board, an electronic signature receiving device, a projector, or various measurement devices (such as devices for measuring water, electricity, gas, or electromagnetic waves). Note that, according to various embodiments of this disclosure, an electronic device may be one or a combination of the above-listed devices. According to some embodiments of this disclosure, the electronic device may be a flexible electronic device. The electronic device disclosed here is not limited to the above-listed devices and may include new electronic devices depending on the development of technology.

In the following description, electronic devices are described with reference to the accompanying drawings, according to various embodiments of this disclosure. As used here, the term “user” may denote a human or another device (such as an artificial intelligent electronic device) using the electronic device.

Definitions for other certain words and phrases may be provided throughout this patent document. Those of ordinary skill in the art should understand that in many if not most instances, such definitions apply to prior as well as future uses of such defined words and phrases.

None of the description in this application should be read as implying that any particular element, step, or function is an essential element that must be included in the claim scope. The scope of patented subject matter is defined only by the claims. Moreover, none of the claims is intended to invoke 35 U.S.C. § 112(1) unless the exact words “means for” are followed by a participle. Use of any other term, including without limitation “mechanism,” “module,” “device,” “unit,” “component,” “element,” “member,” “apparatus,” “machine,” “system,” “processor,” or “controller,” within a claim is understood by the Applicant to refer to structures known to those skilled in the relevant art and is not intended to invoke 35 U.S.C. § 112(f).

### BRIEF DESCRIPTION OF THE DRAWINGS

For a more complete understanding of this disclosure and its advantages, reference is now made to the following description taken in conjunction with the accompanying drawings, in which like reference numerals represent like parts:

FIG. 1 illustrates an example network configuration including an electronic device according to this disclosure;

FIG. 2 illustrates an example framework for stress profiling and personalized stress intervention recommendation according to this disclosure;

FIG. 3 illustrates an example stress data profile for one or more stressors experienced by a user according to this disclosure;

FIG. 4 illustrates an example process for stress profiling and personalized stress intervention recommendation based on the framework of FIG. 2 according to this disclosure;

FIGS. 5 and 6 illustrate other example frameworks for stress profiling and personalized stress intervention recommendation according to this disclosure; and

FIG. 7 illustrates an example method for stress profiling and personalized stress intervention recommendation according to this disclosure.

### DETAILED DESCRIPTION

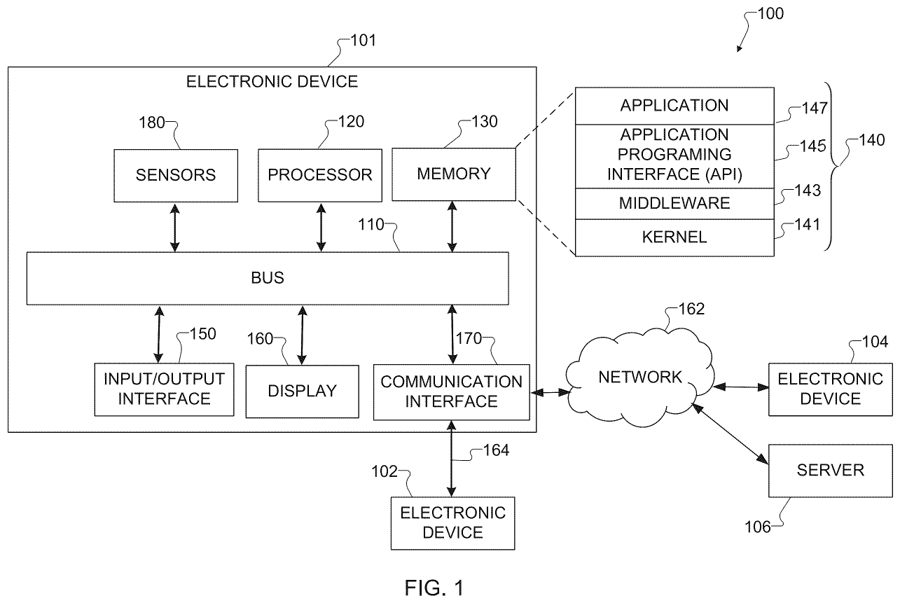

FIGS. 1 through 7, discussed below, and the various embodiments of this disclosure are described with reference to the accompanying drawings. However, it should be appreciated that this disclosure is not limited to these embodiments, and all changes and/or equivalents or replacements thereto also belong to the scope of this disclosure.

As discussed above, stress is a leading cause of physical and psychological conditions in modern life. In the United States, 77% of people report regularly experiencing physical symptoms caused by stress, and 73% of people regularly experience psychological symptoms caused by stress. Among other medical issues, unchecked stress is associated with brain function complications, compromised immune system functions, and cardiovascular and gastrointestinal complications. The annual cost of stress-related healthcare and lost productivity in the United States is currently estimated to be 300 billion.

Stressors can come from a variety of different sources (such as social stress, deadline stress, traffic stress, and the like), and a given individual can have a unique set of responses to these stressors. For example, heart rate variability (HRV) is sensitive to changes in autonomic nervous system activity associated with stress. Stress-induced respiratory reactions can include symptoms such as hyperventilation. In addition, stress exposure can result in increased intestinal temperature and reduced skin temperature at distal locations, such as the fingertips. The changes in these parameters are variable due to factors such as fitness level, age, and severity of the stressor. Many individuals may not be fully aware of the context and extent of the stresses they undergo. Moreover, many individuals may be unlikely to know the amount of stress they experience in any given day or time period. This makes prescribing appropriate interventions difficult.

There are a number of interventions that can alleviate or reduce the occurrence of stress events, and each type of intervention can have varying levels of convenience and efficacy for each user. Some interventions may require a user to be mindful of his or her stress level and take active steps to utilize the intervention, which can be a mental barrier to habitually using them. Moreover, different individuals can have different responses to the same stressors and different responses to the same stress interventions, thus making it challenging to detect stress and recommend an appropriate intervention. Ultimately, individuals may be unsure of the most effective way to alleviate the effect of stress and may rely on trial and error to find an appropriate intervention that works for them. In addition, some individuals may find it challenging to track the effectiveness of interventions and know if they are applying it appropriately, which can lead to low compliance and lack of use.

This disclosure provides systems and methods for stress profiling and personalized stress intervention recommendation. As described in more detail below, the disclosed systems and methods receive stress data associated with a user and determine stress profile features associated with the user. Using the stress profile features, a trained stress profile identification machine learning model selects a stress profile for association with the user, and a trained stress intervention recommendation machine learning model can select a stress intervention activity for the user based on the stress profile and context data. Compared to prior techniques, the disclosed embodiments can improve the detection and management of stress for individuals, including those in high-stress settings, such as office workers, medical residents, and military personnel. Note that while some of the embodiments discussed below are described in the context of use in consumer electronic devices (such as smart phones), this is merely one example, and it will be understood that the principles of this disclosure may be implemented in any number of other suitable contexts.

FIG. 1 illustrates an example network configuration 100 including an electronic device according to this disclosure. The embodiment of the network configuration 100 shown in FIG. 1 is for illustration only. Other embodiments of the network configuration 100 could be used without departing from the scope of this disclosure.

According to embodiments of this disclosure, an electronic device 101 is included in the network configuration 100. The electronic device 101 can include at least one of a bus 110, a processor 120, a memory 130, an input/output (I/O) interface 150, a display 160, a communication interface 170, or a sensor 180. In some embodiments, the electronic device 101 may exclude at least one of these components or may add at least one other component. The bus 110 includes a circuit for connecting the components 120-180 with one another and for transferring communications (such as control messages and/or data) between the components.

The processor 120 includes one or more of a central processing unit (CPU), an application processor (AP), or a communication processor (CP). The processor 120 is able to perform control on at least one of the other components of the electronic device 101 and/or perform an operation or data processing relating to communication. In some embodiments, the processor 120 can be a graphics processor unit (GPU). As described in more detail below, the processor 120 may perform one or more operations for stress profiling and personalized stress intervention recommendation.

The memory 130 can include a volatile and/or non-volatile memory. For example, the memory 130 can store commands or data related to at least one other component of the electronic device 101. According to embodiments of this disclosure, the memory 130 can store software and/or a program 140. The program 140 includes, for example, a kernel 141, middleware 143, an application programming interface (API) 145, and/or an application program (or “application”) 147. At least a portion of the kernel 141, middleware 143, or API 145 may be denoted an operating system (OS).

The kernel 141 can control or manage system resources (such as the bus 110, processor 120, or memory 130) used to perform operations or functions implemented in other programs (such as the middleware 143, API 145, or application 147). The kernel 141 provides an interface that allows the middleware 143, the API 145, or the application 147 to access the individual components of the electronic device 101 to control or manage the system resources. The application 147 may support one or more functions for stress profiling and personalized stress intervention recommendation as discussed below. These functions can be performed by a single application or by multiple applications that each carry out one or more of these functions. The middleware 143 can function as a relay to allow the API 145 or the application 147 to communicate data with the kernel 141, for instance. A plurality of applications 147 can be provided. The middleware 143 is able to control work requests received from the applications 147, such as by allocating the priority of using the system resources of the electronic device 101 (like the bus 110, the processor 120, or the memory 130) to at least one of the plurality of applications 147. The API 145 is an interface allowing the application 147 to control functions provided from the kernel 141 or the middleware 143. For example, the API 145 includes at least one interface or function (such as a command) for filing control, window control, image processing, or text control.

The I/O interface 150 serves as an interface that can, for example, transfer commands or data input from a user or other external devices to other component(s) of the electronic device 101. The I/O interface 150 can also output commands or data received from other component(s) of the electronic device 101 to the user or the other external device.

The display 160 includes, for example, a liquid crystal display (LCD), a light emitting diode (LED) display, an organic light emitting diode (OLED) display, a quantum-dot light emitting diode (QLED) display, a microelectromechanical systems (MEMS) display, or an electronic paper display. The display 160 can also be a depth-aware display, such as a multi-focal display. The display 160 is able to display, for example, various contents (such as text, images, videos, icons, or symbols) to the user. The display 160 can include a touchscreen and may receive, for example, a touch, gesture, proximity, or hovering input using an electronic pen or a body portion of the user.

The communication interface 170, for example, is able to set up communication between the electronic device 101 and an external electronic device (such as a first electronic device 102, a second electronic device 104, or a server 106). For example, the communication interface 170 can be connected with a network 162 or 164 through wireless or wired communication to communicate with the external electronic device. The communication interface 170 can be a wired or wireless transceiver or any other component for transmitting and receiving signals.

The wireless communication is able to use at least one of, for example, long term evolution (LTE), long term evolution-advanced (LTE-A), 5th generation wireless system (5G), millimeter-wave or 60 GHz wireless communication, Wireless USB, code division multiple access (CDMA), wideband code division multiple access (WCDMA), universal mobile telecommunication system (UMTS), wireless broadband (WiBro), or global system for mobile communication (GSM), as a cellular communication protocol. The wired connection can include, for example, at least one of a universal serial bus (USB), high definition multimedia interface (HDMI), recommended standard 232 (RS-232), or plain old telephone service (POTS). The network 162 or 164 includes at least one communication network, such as a computer network (like a local area network (LAN) or wide area network (WAN)), Internet, or a telephone network.

The electronic device 101 further includes one or more sensors 180 that can meter a physical quantity or detect an activation state of the electronic device 101 and convert metered or detected information into an electrical signal. For example, one or more sensors 180 include one or more cameras or other imaging sensors for capturing images of scenes. The sensor(s) 180 can also include one or more buttons for touch input, a gesture sensor, a gyroscope or gyro sensor, an air pressure sensor, a magnetic sensor or magnetometer, an acceleration sensor or accelerometer, a grip sensor, a proximity sensor, a color sensor (such as a red green blue (RGB) sensor), a bio-physical sensor, a temperature sensor, a humidity sensor, an illumination sensor, an ultraviolet (UV) sensor, an electromyography (EMG) sensor, an electroencephalogram (EEG) sensor, an electrocardiogram (ECG) sensor, an infrared (IR) sensor, an ultrasound sensor, an iris sensor, or a fingerprint sensor. The sensor(s) 180 can further include an inertial measurement unit, which can include one or more accelerometers, gyroscopes, and other components. In addition, the sensor(s) 180 can include a control circuit for controlling at least one of the sensors included here. Any of these sensor(s) 180 can be located within the electronic device 101.

The first external electronic device 102 or the second external electronic device 104 can be a wearable device or an electronic device-mountable wearable device (such as an HMD). When the electronic device 101 is mounted in the electronic device 102 (such as the HMD), the electronic device 101 can communicate with the electronic device 102 through the communication interface 170. The electronic device 101 can be directly connected with the electronic device 102 to communicate with the electronic device 102 without involving with a separate network. The electronic device 101 can also be an augmented reality wearable device, such as eyeglasses, that include one or more imaging sensors.

The first and second external electronic devices 102 and 104 and the server 106 each can be a device of the same or a different type from the electronic device 101. According to certain embodiments of this disclosure, the server 106 includes a group of one or more servers. Also, according to certain embodiments of this disclosure, all or some of the operations executed on the electronic device 101 can be executed on another or multiple other electronic devices (such as the electronic devices 102 and 104 or server 106). Further, according to certain embodiments of this disclosure, when the electronic device 101 should perform some function or service automatically or at a request, the electronic device 101, instead of executing the function or service on its own or additionally, can request another device (such as electronic devices 102 and 104 or server 106) to perform at least some functions associated therewith. The other electronic device (such as electronic devices 102 and 104 or server 106) is able to execute the requested functions or additional functions and transfer a result of the execution to the electronic device 101. The electronic device 101 can provide a requested function or service by processing the received result as it is or additionally. To that end, a cloud computing, distributed computing, or client-server computing technique may be used, for example. While FIG. 1 shows that the electronic device 101 includes the communication interface 170 to communicate with the external electronic device 104 or server 106 via the network 162 or 164, the electronic device 101 may be independently operated without a separate communication function according to some embodiments of this disclosure.

The server 106 can include the same or similar components 110-180 as the electronic device 101 (or a suitable subset thereof). The server 106 can support to drive the electronic device 101 by performing at least one of operations (or functions) implemented on the electronic device 101. For example, the server 106 can include a processing module or processor that may support the processor 120 implemented in the electronic device 101. As described in more detail below, the server 106 may perform one or more operations to support techniques for stress profiling and personalized stress intervention recommendation.

Although FIG. 1 illustrates one example of a network configuration 100 including an electronic device 101, various changes may be made to FIG. 1. For example, the network configuration 100 could include any number of each component in any suitable arrangement. In general, computing and communication systems come in a wide variety of configurations, and FIG. 1 does not limit the scope of this disclosure to any particular configuration. Also, while FIG. 1 illustrates one operational environment in which various features disclosed in this patent document can be used, these features could be used in any other suitable system.

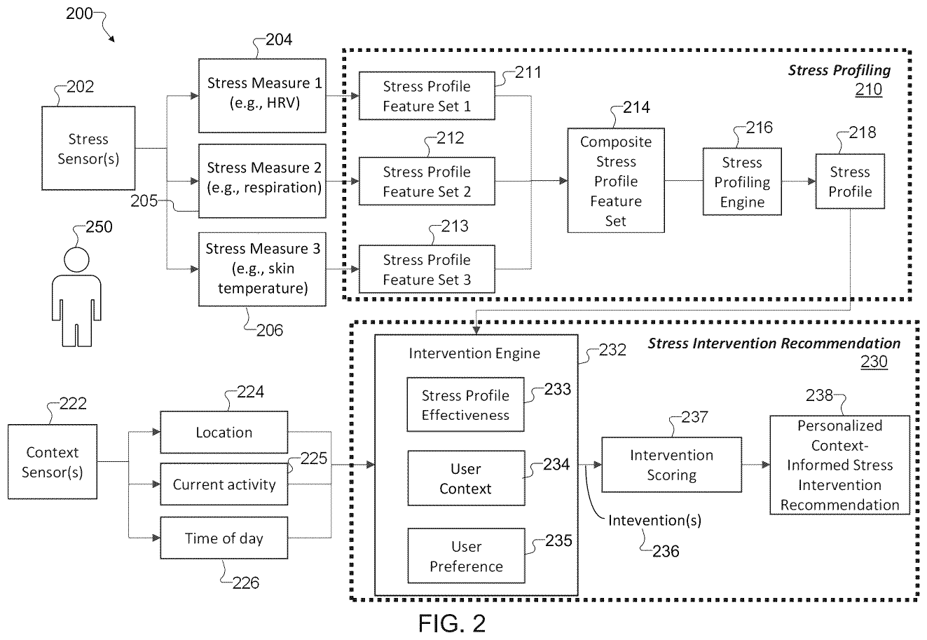

FIG. 2 illustrates an example framework 200 for stress profiling and personalized stress intervention recommendation according to this disclosure. For ease of explanation, the framework 200 is described as involving the use of one or more components of the electronic device 101 described above. However, this is merely one example, and the framework 200 could be implemented using any additional or other suitable device(s), such as the server 106.

As shown in FIG. 2, the framework 200 includes information received from one or more stress sensors 202 and one or more context sensors 222. Any of the stress sensor(s) 202 or context sensor(s) 222 may be a part of, coupled to, or otherwise associated with the electronic device 101. In some embodiments, the stress sensor(s) 202 or context sensor(s) 222 may represent (or be represented by) the sensors 180 of FIG. 1. Also, in some embodiments, one or more of the stress sensor(s) 202 or context sensor(s) 222 may be physically separated from, but communicatively coupled to, the electronic device 101. Further, in some embodiments, one or more of the stress sensor(s) 202 may be positioned in or on a smart watch or earbuds. In addition, in some embodiments, one or more of the context sensor(s) 222 may be positioned in or on a smart phone. Of course, other arrangements and positions of the stress sensor(s) 202 and context sensor(s) 222 are possible and within the scope of this disclosure.

The one or more stress sensors 202 are capable of measuring or detecting stress-related data associated with a user 250. For example, the stress sensor(s) 202 can measure or detect heart rate or HRV of the user 250, respiration of the user 250, skin temperature of the user 250, blood pressure of the user 250, and the like. These measurements can be obtained continuously, intermittently, on demand, according to a schedule, or at any other suitable time(s). The stress sensors(s) 202 can output multiple stress-related measurements 204-206, which may include HRV-based measurements 204, respiration-based measurements 205, and skin temperature-based measurements 206. While the framework 200 is shown with three stress-related measurements 204-206, this is merely one example, and the framework 200 could include other numbers of stress-related measurements obtained by the stress sensor(s) 202.

The electronic device 101 obtains the stress-related measurements 204-206 and performs a stress profiling operation 210 to determine a stress profile 218 for the user 250. In the stress profiling operation 210, the electronic device 101 determines a stress profile feature set 211-213 based on each set of obtained stress-related measurements 204-206. For instance, the electronic device 101 may determine a stress profile feature set 211 based on the HRV-based measurements 204, a stress profile feature set 212 based on the respiration-based measurements 205, and a stress profile feature set 213 based on the skin temperature-based measurements 206. Each stress profile feature set 211-213 includes one or more stress profile features that are related to a stress response of the user 250. In some embodiments, the stress profile features included in each stress profile feature set 211-213 include sympathetic nervous system-parasympathetic nervous system (SNS-PNS) balance, recovery speed, and heterogeneity of stress response. Each of these features is described below.

SNS-PNS Balance

SNS-PNS balance refers to the balance between sympathetic nervous system activity of the user 250 and parasympathetic nervous system activity of the user 250. In most individuals, stress response is caused by a relative increase in the gap between SNS activity and PNS activity. A stress response can manifest as an increase in SNS, a decrease in PNS, or a combination of both depending on the individual and the context.

For each sensing modality (such as HRV, respiration, skin temperature, and the like), there are a number of derived features that respectively reflect SNS and PNS activity. For example, among HRV features, high frequency (HF) band (such as 0.15-0.4 Hz) features are often associated with PNS activity, and low frequency (LF) band (such as 0.04-0.15 Hz) features are often associated with SNS activity. In some cases, for a given stress event, the SNS-PNS balance can be represented as a ratio ρ, which may be determined as follows.

$$ρ = \frac{\frac{1}{{N}_{α}} {\sum }_{i = 1}^{{N}_{α}} ( \frac{{α}_{i} - {μ}_{i}^{α}}{{μ}_{i}^{α}} )}{\frac{1}{{N}_{β}} {\sum }_{i = 1}^{{N}_{β}} ( \frac{{β}_{i} - {μ}_{i}^{β}}{{μ}_{i}^{β}} )}$$

Here, N_{α} represents the number of features related to SNS activity, N_{β} represents the number of features related to PNS activity, αᵢ is an i^{th} SNS feature value during the stress event, βᵢ is an i^{th} PNS feature value during the stress event, μᵢ^{α} is the average value of the i^{th} SNS feature during non-stress periods, and μᵢ^{β} is the average value of the i^{th} PNS feature during non-stress periods.

Recovery Speed

When a subject (such as the user 250) is undergoing stress, one or more biomarkers (such as the stress-related measurements 204-206) obtained from sensors (such as the sensors 202) may become different from their “baseline” values. As used here, baseline values refer to values of biomarkers when a subject is not in a state of stress or is in a state of typical stress (since many individuals typically experience at least a minimal amount of stress at most times). Biomarkers are often elevated in response to stressors (such as in the case of heart rate), although other biomarkers may be lowered in response to stressors (such as skin temperature). In the framework 200, the baseline values for the stress-related measurements 204-206 can be obtained when the user 250 is wearing the stress sensor(s) 202 during normal periods when the user 250 is not undergoing stress or is only experiencing typical stress levels. For example, in some embodiments, one day of non-stress or low-stress data may be sufficient to determine the baseline values, although other time periods are possible and within the scope of this disclosure.

The recovery speed is represented by the time it takes for all biomarker values to return to their baseline values and remain there for a predetermined amount of time. In some embodiments, some or all of the biomarkers may need to remain at their baseline values for a specified time period, such as at least thirty minutes, to be considered a full recovery and the end of the stressor. Of course, time periods other than thirty minutes are possible and within the scope of this disclosure.

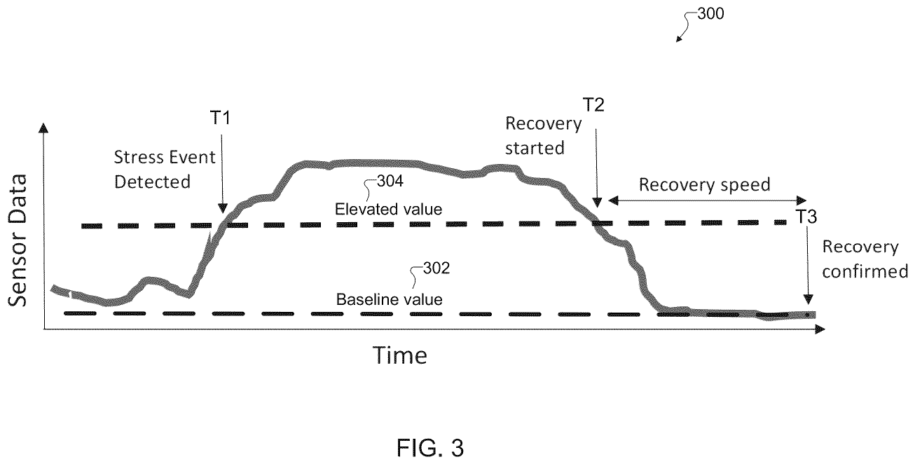

FIG. 3 shows an example stress data profile 300 for one or more stressors experienced by the user 250 according to this disclosure. As shown in FIG. 3, stress-related measurements (such as one of the stress-related measurements 204-206) from a stress sensor 202 are obtained for a period of time for the user 250 and recorded as the stress data profile 300. At time T1, a stress event is detected based on the biomarker value rising from the baseline value 302 to an elevated value 304. The biomarker value stays elevated for a period of time until time T2, when the biomarker value falls below the elevated value 304. This indicates that recovery has started. At time T3, it is determined that the biomarker value has been at approximately the baseline value for a predetermined amount of time, and recovery is confirmed. The elapsed time between T2 and T3 is considered to be the recovery speed.

Heterogeneity of Stress Response

The heterogeneity of a user's stress response refers to how much the user's stress response changes over time for different occurrences of a stress event. In some embodiments, the heterogeneity of a user's stress response is estimated by the standard deviation of the peaks of biomarkers across multiple stress events, the number of different biomarkers that show statistically significant change across multiple stress events, any other suitable factors, or a combination of these. These factors can be normalized to account for varying availability of different sensors among various users and for the same user at various times of the day.

Once the stress profile feature sets 211-213 have been determined, the electronic device 101 uses the stress profile feature sets 211-213 to generate a composite stress profile feature set 214. There are various ways to generate the composite stress profile feature set 214 from the stress profile feature sets 211-213, and any suitable technique or algorithm can be used. In some embodiments, the electronic device 101 averages the stress profile feature sets 211-213 in a weighted manner to compute the composite stress profile feature set 214. That is, each feature (such as SNS-PNS balance, recovery speed, and heterogeneity of stress response) forming the stress profile feature sets 211-213 can be assigned a particular weight before the averaging is performed. In some cases, the weights for the features are pre-determined and fixed based on the relative importance of each feature to stress measurement. For example, for many individuals, HRV is more related to stress than respiration. Thus, the HRV may be given a higher weight than respiration.

After the composite stress profile feature set 214 is determined, the electronic device 101 provides the composite stress profile feature set 214 as input to a stress profiling engine 216 in order to determine the stress profile 218. The stress profiling engine 216 selects the stress profile 218 from among a group of predetermined candidate stress profiles to represent the stress response of the user 250 in response to various external factors. As discussed above, a stress response can manifest as an increase in SNS, a decrease in PNS, or a combination of both depending on the individual and the context. Individuals can have different stress responses to different stressors (which is sometimes referred to as heterogeneity of stress response). The time to recover from a stressor and return to baseline can vary based on the individual and the type of stressor.

The stress profiling engine 216 includes a stress profile identification machine learning model that can be trained to select the stress profile 218 based on the composite stress profile feature set 214. The stress profile identification model may have any suitable machine learning-based structure, such as a convolution neural network, deep learning network, or other architecture. In some embodiments, the stress profiling engine 216 can use a clustering algorithm to assign the user 250 to one of multiple clusters according to the characteristics of the stress response of the user 250 as represented in the composite stress profile feature set 214. Each cluster represents a stress profile 218 that informs stress intervention recommendations. In some embodiments, a week of stress response data (or data over some other time period) is used to place the user 250 in a given cluster, thereby selecting a stress profile 218.

In some embodiments, the stress profile identification model is trained using unsupervised learning to cluster multiple users in multiple clusters based on stress profile features of the users. For example, the training data on which the stress profile identification model is trained can be collected from multiple sensors associated with the multiple users while the multiple users experience various stressors. The multiple users from which the training data is generated can be selected to represent particular groups (such as demographic groups, health condition groups, geographic groups, and the like) or can be selected to represent a wide variety of users. The training data (which may include HRV-based features, respiration-based features, skin temperature-based features, and the like) can be used to train the stress profile identification model to identify the stress profile to which each of the multiple users belongs. As a particular example, training data can be obtained from multiple subjects in a lab setting and other subjects in a free-living scenario, where ground truth information can be provided by reference physiological sensors or self-reports.

Once a stress profile 218 has been determined for the user 250, the electronic device 101 can perform a stress intervention recommendation operation 230 to determine a stress intervention recommendation 238 that is personalized for the user 250 and is informed by the context of the user 250. The stress intervention recommendation operation 230 provides an automatic recommendation of a stress intervention based on categorization of the detected stressor(s). As discussed in greater detail below, the stress intervention recommendation operation 230 balances convenience, personal preferences, and effectiveness based on a current context of the user 250.

The stress intervention recommendation operation 230 uses context information obtained using the one or more context sensors 222. The context sensor(s) 222 measure or detect context-related data 224-226 associated with the user 250. For example, the context sensor(s) 222 can measure or detect location data 224 associated with the user 250, a current activity 225 of the user 250 (which can include an actual activity of the user 250 (such as sitting, walking, running, reading, working, sleeping, and the like) or movement information of the user 250 (such as speed, direction, and the like)), time/date information 226, and the like. These measurements can be obtained continuously, intermittently, on demand, according to a schedule, or at any other suitable time(s). The context sensors(s) 222 can output the context data 224-226 for use in the stress intervention recommendation operation 230. While the framework 200 is shown with three types of context data 224-226, this is merely one example, and the framework 200 could include other numbers and types of context data 224-226 obtained by the context sensor(s) 222.

After the context data 224-226 is determined, the electronic device 101 provides the context data 224-226 as input to an intervention engine 232 in order to determine one or more possible stress intervention activities or techniques (or simply “interventions”) 236 for the user 250. The intervention engine 232 includes a stress intervention recommendation machine learning model that can be trained to select the interventions 236 based on the stress profile 218 of the user 250, the context data 224-226 associated with the user 250, and user preference 235. The stress intervention recommendation model may have any suitable machine learning-based structure, such as a convolution neural network, deep learning network, or other architecture. The intervention engine 232 determines the possible interventions 236 and associates each intervention 236 with multiple factors, such as stress profile effectiveness 233, user context 234, and user preference 235.

Stress Profile Effectiveness

In general, advantageous interventions for a particular stress profile include those interventions that are most effective for that stress profile. Thus, the stress profile effectiveness 233 of each intervention 236 for the stress profile 218 of the user 250 is considered by the intervention engine 232. In some embodiments, the stress profile effectiveness 233 for a particular stress profile 218 can be represented as a score, such as a score from 1 to 5, with 5 being the most effective for the given stress profile 218 (although other scoring schemes are possible).

In some embodiments, the stress intervention recommendation model of the intervention engine 232 is trained using unsupervised learning to identify the most effective intervention(s) 236 for each stress profile 218 identified in the training. In some embodiments, the stress profile effectiveness 233 can be estimated using one or more reference physiological sensors available in during training, such as cardiac output and blood pressure devices. Also, in some embodiments, the interventions 236 that bring measurements from the physiological sensors closer to baseline in a shorter duration can be given a higher effectiveness score. Further, in some embodiments, it may be assumed that the “best fit” intervention 236 identified for each stress profile 218 during training continues to be the most effective intervention 236 for each new stress profile 218 encountered in real world scenarios.

User Context

The user context 234 is a combination of the context data, such as the location data 224 associated with the user 250, the current activity 225 of the user 250, the time/date information 226 of the stress response, and any other suitable context information. The user context 234 can be used to determine an appropriateness of a particular intervention 236. For example, performing breathing exercises in a public setting can be considered inappropriate. Thus, if the user 250 is currently in a public setting at the time of a stress response, it may not be appropriate to recommend that the user 250 perform visible or disruptive breathing exercises. In some embodiments, the intervention engine 232 compares the interventions 236 to the user context 234 to determine an appropriateness score for each intervention 236. For example, the appropriateness score could be from 1 to 5, with 1 indicating that the intervention 236 is least appropriate for the current user context 234 and 5 indicating that the intervention 236 is most appropriate for the user context 234 (although other scoring schemes are possible). Also, in some embodiments, the intervention engine 232 determines the appropriateness scores based on prior knowledge (such as performing breathing exercises in a public setting can be considered inappropriate) and user feedback in prior training data.

User Preference

The user preference 235 indicates a level of preference of the user 250 for a particular intervention 236 in response to a stressor. In some embodiments, the electronic device 101 can track and store, over time, previous stress responses of the user 250 to a stressor in order to learn preferences for interventions 236. For example, the electronic device 101 can track that the user 250 often engages in breathing exercises in response to a stressor, or the electronic device 101 can track that the user 250 ignores recommendations to engage in physical activity in response to a stressor. Thus, the electronic device 101 can assign a high user preference 235 to the breathing exercises and can assign a low user preference 235 to the physical activity. In some embodiments, the user preference 235 could be represented as a score from 1 to 5, with 1 indicating that the intervention 236 is least preferred by the user 250 and 5 indicating that the intervention 236 is most preferred by the user 250 (although other scoring schemes are possible).

Once the intervention engine 232 determines the possible interventions 236 for the user 250, the electronic device 101 performs an intervention scoring operation 237 to rank the possible interventions 236. The intervention scoring operation 237 can take into account the stress profile effectiveness 233 of each intervention 236, the appropriateness of the intervention 236 for the current user context 234, and the user preference 235 for the intervention 236. In some embodiments, the electronic device 101 can use a linear regression model to compute the overall score for an intervention 236, which may be determined as follows.

S=αs₁+βs₂+γs₃

Here, s₁ is the stress profile effectiveness 233 of the intervention 236 for the stress profile 218 of the user 250, s₂ is the appropriateness of the intervention 236 for the current user context 234, s₃ is the user preference 235 for the intervention 236, S is the overall intervention score for the intervention 236, and α, β, γ are weights to be regressed based on prior data. In some embodiments, some or all of the interventions 236 initially have a predetermined default score and are dynamically updated up or down based on the user's real-time choices of intervention over time. With each update, one or more of the weights of the regression model can be updated based on effectiveness of intervention and user feedback.

After the interventions 236 have been ranked, the electronic device 101 makes an intervention recommendation 238 to the user 250. For example, the electronic device 101 can show the intervention recommendation 238 on the display 160 of the electronic device 101, generate one or more audible sounds or words that can be heard by the user, employ haptics, or use any other suitable technique to notify the user 250 of the intervention recommendation 238. As discussed above, the intervention recommendation 238 is personalized for the user 250 and is informed by the context of the user 250. In some embodiments, the electronic device 101 can select the intervention 236 with the highest score as the intervention recommendation 238. Of course, the electronic device 101 can use any other suitable technique for selecting the intervention 236 for the intervention recommendation 238.

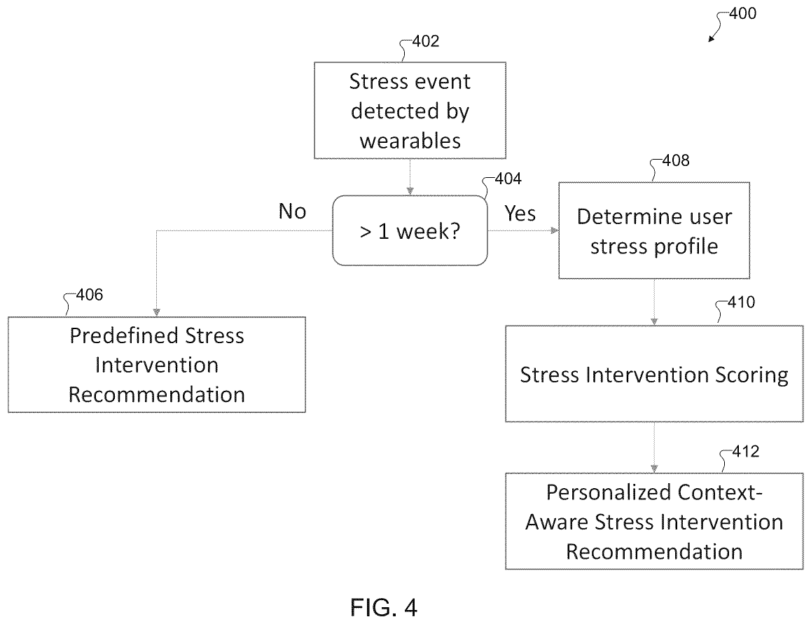

FIG. 4 illustrates an example process 400 for stress profiling and personalized stress intervention recommendation based on the framework 200 of FIG. 2 according to this disclosure. As shown in FIG. 4, at operation 402, the electronic device 101 detects one or more stress events of the user 250 based on information received from the sensors 202 and 222. At operation 404, the electronic device 101 determines how much time has passed since the electronic device 101 started tracking and recording stress events for the user 250. If the amount of time is less than a threshold period of time (such as one week), the electronic device 101 may not have adequate personalized stress intervention data for the user 250. Thus, at operation 406, the electronic device 101 recommends interventions 236 based on a pre-defined list that is not personalized for the user 250.

Over time, the electronic device 101 can learn the preferences of the user 250 based on the user's actions in response to stressors. For example, after one week of detected stress events, the electronic device 101 can learn various stress profiles 218 of the user 250 and can learn the “fit” for each possible intervention 236 for that stress profile 218. For example, the electronic device 101 can learn that guided breathing is the most effective intervention 236 for times when the user 250 exhibits a particular stress profile 218. Thus, at operation 404, if the electronic device 101 determines that the amount time that has passed since the electronic device 101 started tracking and recording stress events for the user 250 is greater than the threshold period of time, the process 400 moves to operation 408, where the electronic device 101 determines the current stress profile 218 for the user 250. At operation 410, the electronic device 101 determines one or more possible interventions 236 and scores the interventions 236 to determine the best one or ones, taking into account the effectiveness score, appropriateness score, and user preference score. At operation 412, the electronic device 101 recommends the best intervention(s) 236 to the user 250.

As a particular example of this, while guided breathing may have the highest effectiveness score among the possible interventions 236 for the current stress profile 218, the electronic device 101 may determine that the user 250 is currently in a public setting on a crowded subway, thus reducing the appropriateness score for this intervention 236. Furthermore, the electronic device 101 may detect that the user 250 is wearing earbuds and has preferred relaxation music in the past to calm down. Hence, the electronic device 101 can determine that the recommended intervention 236 is relaxation music via the earbuds.

Although FIGS. 2 through 4 illustrates one example of a framework 200 for stress profiling and personalized stress intervention recommendation and related details, various changes may be made to FIGS. 2 through 4. For example, in some embodiments, the user 250 can be wearing (or otherwise possess) multiple devices such as smart phone, smart watch, earbuds, etc., and each may be capable of providing information for identifying or delivering an intervention recommendation 238. Also, during the intervention scoring operation 237, the electronic device 101 may additionally consider combinations of simultaneous interventions 236 from the multiple devices, such as haptic feedback from the smart watch at the same time as relaxation music from the earbuds. In some embodiments, the combination of devices with the highest score is provided as the intervention recommendation 238. In the case of multiple devices being capable of providing the same intervention 236 (such as haptic feedback from earbuds or smart watch), the electronic device 101 can choose the most appropriate device for the intervention recommendation 238 based on availability, user context 234, user preference 235, or any other suitable consideration(s).

In some embodiments, the framework 200 is applied to a group of users 250 rather than a single user 250. The group of users 250 may include workers in an office, players on a sports team, or the like. The users 250 in the group may all be wearing the same or similar stress sensors 202 and may be going through the same stress event and could benefit from a collective stress intervention, such as music played over speakers. In such embodiments, the electronic device 101 can provide an intervention recommendation 238 that provides the maximum benefit for the maximum number of people in the group, rather than different personalized interventions for each individual user 250.

While the framework 200 is described with various examples of machine learning models and tasks, other embodiments could include other machine learning models and/or other tasks. Also, various operations shown in FIGS. 2 through 4 could overlap, occur in parallel, occur in a different order, or occur any number of times (including zero times). In addition, the specific operations shown in FIGS. 2 through 4 are examples only, and other techniques could be used to perform each of the operations shown in FIGS. 2 through 4.

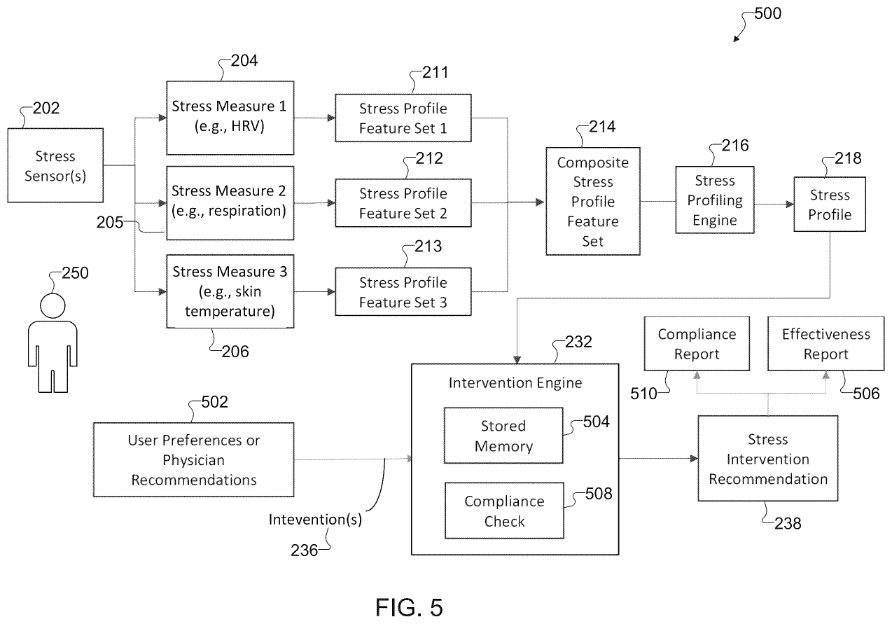

FIG. 5 illustrates another example framework 500 for stress profiling and personalized stress intervention recommendation according to this disclosure. For ease of explanation, the framework 500 is described as involving the use of one or more components of the electronic device 101 described above. However, this is merely one example, and the framework 500 could be implemented using any additional or other suitable device(s), such as the server 106.

As shown in FIG. 5, the framework 500 includes many components that are the same as or similar to corresponding components in the framework 200 of FIG. 2. However, instead of the intervention engine 232 determining a set of possible interventions 236, the possible interventions 236 are determined by the user 250 or by a third party 502, such as a physician or a wellness coach. The intervention engine 232 can store the possible interventions 236 in a memory 504 and provide one or more of the interventions 236 as the intervention recommendation 238 in response to the current stress profile 218 of the user 250.

The electronic device 101 can also learn information to provide a better intervention recommendation 238, similar to the framework 200. For example, the electronic device 101 can obtain input from the user 250 on the user's difficulty in performing a given intervention 236 (such as breathing exercises). Over time, the electronic device 101 can learn the user's preferred intervention 236 for each given stressor, which can be stored in the memory 504. Once the learning is established, whenever the electronic device 101 detects elevated stress, the electronic device 101 can automatically provide an intervention recommendation 238 from the memory 504. Once the user 250 performs the intervention(s) of the intervention recommendation 238, an effectiveness report 506 can be generated for review by the physician or wellness coach.

In addition, the intervention engine 232 can perform one or more compliance checks 508, in which the intervention engine 232 determines whether the user 250 complies with the provided intervention recommendation 238. Once the user 250 performs, partially performs, or does not perform the intervention(s) of the intervention recommendation 238, a compliance report 510 can be generated for review by the physician, wellness coach, or other personnel.

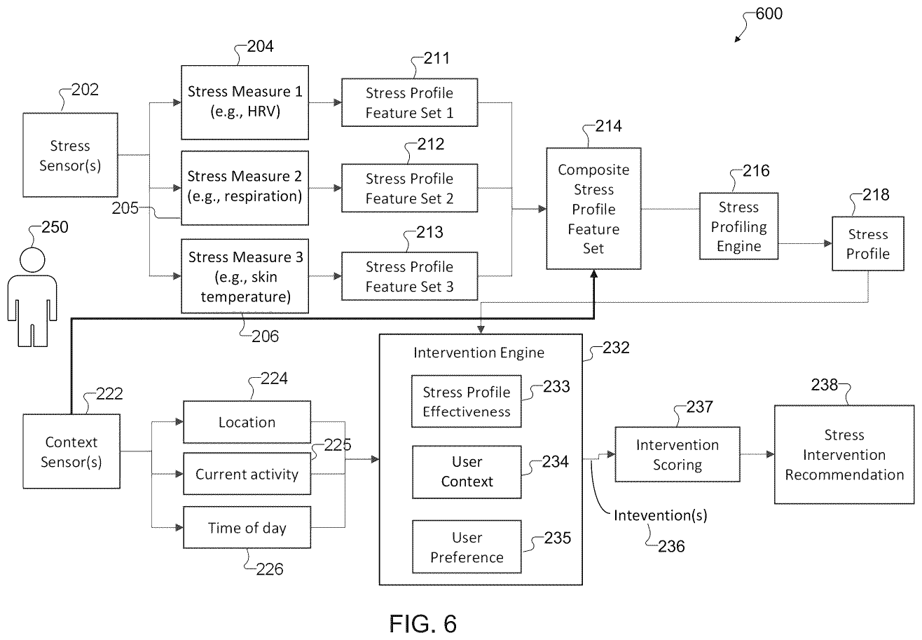

FIG. 6 illustrates yet another example framework 600 for stress profiling and personalized stress intervention recommendation according to this disclosure. For ease of explanation, the framework 600 is described as involving the use of one or more components of the electronic device 101 described above. However, this is merely one example, and the framework 600 could be implemented using any additional or other suitable device(s), such as the server 106.

As shown in FIG. 6, the framework 600 includes many components that are the same as or similar to corresponding components in the framework 200 of FIG. 2. However, in the framework 600, context data 224-226 is obtained from the context sensor(s) 222 and used in a feedback loop, which can be used to update the stress profile 218 of the user 250. For example, the context data 224-226 can be provided as an input when the electronic device 101 determines the composite stress profile feature set 214. As a particular example, if the electronic device 101 learns, using the context data 224-226, that a particular location or activity is a source of stress for the user 250 (such as anxiety being within large crowds, driving in traffic, etc.), this information can help determine a better stress profile 218 for the user 250.

Although FIGS. 5 and 6 illustrate examples of other frameworks 500, 600 for stress profiling and personalized stress intervention recommendation and related details, various changes may be made to FIGS. 5 and 6. For example, various operations shown in FIGS. 5 and 6 could overlap, occur in parallel, occur in a different order, or occur any number of times (including zero times). In addition, the specific operations shown in FIGS. 5 and 6 are examples only, and other techniques could be used to perform each of the operations shown in FIGS. 5 and 6.

Note that the operations and functions shown in or described with respect to FIGS. 2 through 6 can be implemented in an electronic device 101, server 106, or other device in any suitable manner. For example, in some embodiments, the operations and functions shown in or described with respect to FIGS. 2 through 6 can be implemented or supported using one or more software applications or other software instructions that are executed by the processor 120 of the electronic device 101, server 106, or other device. In other embodiments, at least some of the operations and functions shown in or described with respect to FIGS. 2 through 6 can be implemented or supported using dedicated hardware components. In general, the operations and functions shown in or described with respect to FIGS. 2 through 6 can be performed using any suitable hardware or any suitable combination of hardware and software/firmware instructions.

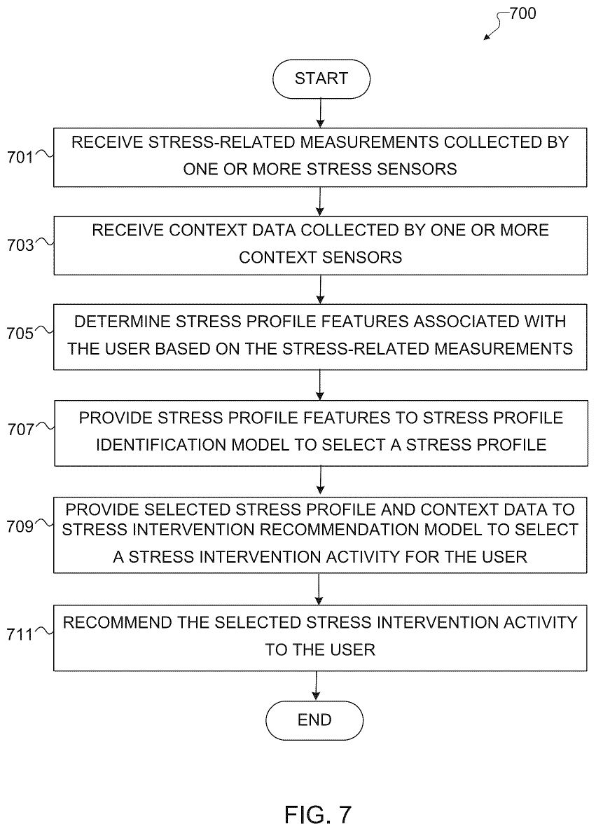

FIG. 7 illustrates an example method 700 for stress profiling and personalized stress intervention recommendation according to this disclosure. For ease of explanation, the method 700 shown in FIG. 7 is described as involving the use of the electronic device 101 shown in FIG. 1 and one or more of the frameworks 200, 500, and 600 shown in FIGS. 2, 5, and 6. However, the method 700 shown in FIG. 7 could be used with any other suitable electronic device and any suitable framework.

As shown in FIG. 7, one or more stress-related measurements collected by one or more stress sensors are received at step 701. This could include, for example, the electronic device 101 receiving stress-related measurements 204-206 associated with the user 250, which are collected by one or more stress sensors 202. The stress-related measurements represent one or more physiological responses of a user to a stressor. Context data collected by one or more context sensors is received at step 703. This could include, for example, the electronic device 101 receiving context data 224-226 collected by one or more context sensors 222. The context data represents a context associated with the user.

One or more stress profile features associated with the user are determined at step 705 based on the stress-related measurements. This could include, for example, the electronic device 101 determining stress profile feature sets 211-213 associated with the user 250 based on the stress-related measurements 204-206. The stress profile features are provided to a trained stress profile identification machine learning model in order to select a stress profile from among multiple candidate stress profiles for association with the user at step 707. This could include, for example, the electronic device 101 generating the composite stress profile feature set 214 from the stress profile feature sets 211-213 and providing the composite stress profile feature set 214 to the stress profiling engine 216 to select a stress profile 218 for association with the user 250.

The selected stress profile and the context data are provided to a trained stress intervention recommendation machine learning model in order to select a stress intervention activity for the user at step 709. This could include, for example, the electronic device 101 providing the selected stress profile 218 and the context data 224-226 to the intervention engine 232 to select a stress intervention 236 for the user 250. The selected stress intervention activity is recommended to the user at step 711. This could include, for example, the electronic device 101 providing the stress intervention recommendation 238 to the user 250.

Although FIG. 7 illustrates one example of a method 700 for stress profiling and personalized stress intervention recommendation, various changes may be made to FIG. 7. For example, while shown as a series of steps, various steps in FIG. 7 could overlap, occur in parallel, occur in a different order, or occur any number of times.

Although this disclosure has been described with reference to various example embodiments, various changes and modifications may be suggested to one skilled in the art. It is intended that this disclosure encompass such changes and modifications as fall within the scope of the appended claims.

What is claimed is:

1. A method comprising:
receiving stress-related measurements collected by one or more stress sensors, the stress-related measurements representing one or more physiological responses of a user to a stressor;
receiving context data collected by one or more context sensors, the context data representing a context associated with the user;
determining stress profile features associated with the user based on the stress-related measurements;
providing the stress profile features to a trained stress profile identification machine learning model to select a stress profile from among multiple candidate stress profiles for association with the user;
providing the selected stress profile and the context data to a trained stress intervention recommendation machine learning model to select a stress intervention activity for the user; and
recommending the selected stress intervention activity to the user.

2. The method of claim 1, wherein the trained stress profile identification machine learning model is trained using unsupervised learning to cluster multiple prior users into multiple clusters based on stress profile features of the prior users, each cluster associated with at least one of the multiple candidate stress profiles.

3. The method of claim 2, wherein the stress profile features comprise at least one of: sympathetic nervous system-parasympathetic nervous system balance, recovery speed, and heterogeneity of stress responses of the user.

4. The method of claim 1, wherein the trained stress intervention recommendation machine learning model is configured to select the stress intervention activity for the user based on at least one of: an effectiveness of the stress intervention activity for the selected stress profile, an appropriateness of the stress intervention activity in a current environment, and a user preference.

5. The method of claim 1, wherein:
the one or more stress sensors are positioned in or on at least one of: a smart watch and earbuds; and
the one or more context sensors are positioned in or on a smart phone.

6. The method of claim 1, wherein the one or more physiological responses of the user to the stressor comprise a change in at least one of: blood pressure, heart rate, skin temperature, respiration, and heart rate variability.

7. The method of claim 1, wherein the context associated with the user comprises at least one of: a location of the user, a current activity of the user, and a current time of day.

8. An electronic device comprising:
at least one memory configured to store instructions; and
at least one processing device configured when executing the instructions to:
receive stress-related measurements collected by one or more stress sensors, the stress-related measurements representing one or more physiological responses of a user to a stressor;
receive context data collected by one or more context sensors, the context data representing a context associated with the user;
determine stress profile features associated with the user based on the stress-related measurements;
provide the stress profile features to a trained stress profile identification machine learning model to select a stress profile from among multiple candidate stress profiles for association with the user;
provide the selected stress profile and the context data to a trained stress intervention recommendation machine learning model to select a stress intervention activity for the user; and
recommend the selected stress intervention activity to the user.

9. The electronic device of claim 8, wherein the trained stress profile identification machine learning model is trained using unsupervised learning to cluster multiple prior users into multiple clusters based on stress profile features of the prior users, each cluster associated with at least one of the multiple candidate stress profiles.

10. The electronic device of claim 9, wherein the stress profile features comprise at least one of: sympathetic nervous system-parasympathetic nervous system balance, recovery speed, and heterogeneity of stress responses of the user.

11. The electronic device of claim 8, wherein the trained stress intervention recommendation machine learning model is configured to select the stress intervention activity for the user based on at least one of: an effectiveness of the stress intervention activity for the selected stress profile, an appropriateness of the stress intervention activity in a current environment, and a user preference.

12. The electronic device of claim 8, wherein:
the one or more stress sensors are positioned in or on at least one of: a smart watch and earbuds; and
the one or more context sensors are positioned in or on a smart phone.

13. The electronic device of claim 8, wherein the one or more physiological responses of the user to the stressor comprise a change in at least one of: blood pressure, heart rate, skin temperature, respiration, and heart rate variability.

14. The electronic device of claim 8, wherein the context associated with the user comprises at least one of: a location of the user, a current activity of the user, and a current time of day.

15. A non-transitory machine-readable medium containing instructions that when executed cause at least one processor of an electronic device to:
receive stress-related measurements collected by one or more stress sensors, the stress-related measurements representing one or more physiological responses of a user to a stressor;
receive context data collected by one or more context sensors, the context data representing a context associated with the user;
determine stress profile features associated with the user based on the stress-related measurements;
provide the stress profile features to a trained stress profile identification machine learning model to select a stress profile from among multiple candidate stress profiles for association with the user;
provide the selected stress profile and the context data to a trained stress intervention recommendation machine learning model to select a stress intervention activity for the user; and
recommend the selected stress intervention activity to the user.

16. The non-transitory machine-readable medium of claim 15, wherein the trained stress profile identification machine learning model is trained using unsupervised learning to cluster multiple prior users into multiple clusters based on stress profile features of the prior users, each cluster associated with at least one of the multiple candidate stress profiles.

17. The non-transitory machine-readable medium of claim 16, wherein the stress profile features comprise at least one of: sympathetic nervous system-parasympathetic nervous system balance, recovery speed, and heterogeneity of stress responses of the user.

18. The non-transitory machine-readable medium of claim 15, wherein the trained stress intervention recommendation machine learning model is configured to select the stress intervention activity for the user based on at least one of: an effectiveness of the stress intervention activity for the selected stress profile, an appropriateness of the stress intervention activity in a current environment, and a user preference.

19. The non-transitory machine-readable medium of claim 15, wherein:
the one or more stress sensors are positioned in or on at least one of: a smart watch and earbuds; and
the one or more context sensors are positioned in or on a smart phone.

20. The non-transitory machine-readable medium of claim 15, wherein the one or more physiological responses of the user to the stressor comprise a change in at least one of: blood pressure, heart rate, skin temperature, respiration, and heart rate variability.
